(19) United States
(12) Patent Application Publication
(10) Pub. No.: US 2021/0361698 A1
(43) Pub. Date: Nov. 25, 2021

(54) COMPOSITION FOR IMPROVING INTESTINAL BARRIER FUNCTION

(71) Applicant: SUNTORY HOLDINGS LIMITED, Osaka-shi, Osaka (JP)

(72) Inventors: Ayuta Funaki, Soraku-gun (JP); Naoki Kasajima, Soraku-gun (JP); Hideyuki Arie, Soraku-gun (JP)

(73) Assignee: SUNTORY HOLDINGS LIMITED, Osaka-shi, Osaka (JP)

(21) Appl. No.: 16/957,529

(22) PCT Filed: Dec. 26, 2018
(86) PCT No.: PCT/JP2018/047863

§ 371 Date: Jun. 24, 2020

(30) Foreign Application Priority Data
Dec. 27, 2017 (JP) 2017-251996

(51) Int. Cl. A61K 31/765; A61K 36/87; A23L 33/10; A61P 1/00

(52) U.S. Cl. CPC A61K 31/765; A61P 1/00; A23L 33/10; A61K 36/87

(57) ABSTRACT

An object of the present invention is to provide a composition for improving intestinal barrier function capable of improving intestinal barrier function. The present invention relates to a composition for improving intestinal barrier function, containing a flavan-3-ol polymer having a weight-average molecular weight of 4500 to 50000 as an active ingredient.

### TECHNICAL FIELD

The present invention relates to a composition for improving intestinal barrier function. The present invention also relates to a method for improving intestinal barrier function, and use of a flavan-3-ol polymer for improving intestinal barrier function.

### BACKGROUND ART

In recent years, awareness with regard to intestinal health has increased, and a large number of intestine-related functional foods have been also sold. An intestinal function mainly includes a nutrient absorption function and a barrier function (intestinal barrier function) which prevents the intrusion (permeation) of toxic substances. Among these, it has become clear that the intestinal barrier function is deeply involved with chronic inflammation diseases increasing with aging.

Underneath intestinal epithelial cells, there exist a great number of immune cells such as macrophages, dendritic cells, T-cells, and B-cells. Typically, the intestinal epithelial cells are tightly joined to each other by a structure referred to as a tight junction, so that high molecular weight substances are strictly controlled so as not to pass through intercellular spaces. The intestinal epithelial cells contain transporters for removing hydrophobic foreign substances from the cells. The tight junction structure, the transporters and the like are responsible for the intestinal barrier function which prevents the intrusion of foreign substances. However, when the intestinal barrier is damaged by aging, neglect of health in life, stress and the like, leading to an increase in intestinal permeability, high molecular weight substances such as intestinal bacteria and their bacterial ingredients, which are present in the intestine, penetrate through intercellular spaces into the body and stimulate immune cells to release inflammatory cytokine, thereby inducing inflammation. As a result, various disease states caused by chronic inflammation such as inflammatory bowel diseases in the intestine, non-alcoholic fatty liver diseases (NAFLD) in the liver, arteriosclerosis-induced symptoms in the blood vessel, generalized diabetes, abnormal lipid metabolism, and autoimmune diseases are considered to be induced. An undigested substance is assumed to intrude out of the intestine to induce allergies.

As described above, the reduction of the intestinal barrier function may cause various diseases or the like. From such a viewpoint, searches for materials which can improve the intestinal barrier function have been attempted. An epithelial cell growth factor (EGF) has been known to promote the maturation of the intestinal epithelial cells, to enhance a barrier function. However, only a small amount of the EGF which is cytokine exists in the living body, so that it is not preferable to use the EGF as a material which improves the intestinal barrier function in respects of economic efficiency and safety. Non-Patent Literature 1 discloses that flavonoid such as quercetin promotes the formation of a tight junction or the like to prevent chronic inflammation. Patent Literature 1 discloses an absorption depressant which contains one or two or more selected from lindane, star anise, marnie, tea, black tea, or treated products thereof as active ingredient(s). Patent Literature 2 discloses that hexapeptide of a specific sequence and tryptophan have absorption suppressive activity of allergen. Patent Literature 3 discloses a supplement to be administered enterally to maintain or restore the intestinal barrier of the intestine, including a combination of glutamine, a substance having antioxidant activity, and a short-chain fatty acid.

### CITATION LIST

Patent Literature
Patent Literature 1: JP 2002-193819 A
Patent Literature 2: JP 2002-257814 A
Patent Literature 3: JP 2004-513912 T
   Non-Patent Literature
Non-Patent literature 1: Suzuki T. et al, The Journal of Nutritional Biochemistry. 2011 May, Vol. 22(5), p.401-408

### SUMMARY OF INVENTION

### Technical Problem

An object of the present invention is to provide a composition for improving intestinal barrier function, the composition capable of improving intestinal barrier function.

### Solution to Problem

In order to solve the above problems, the present inventors intensively studied, and tried to solve the problems by adding inflammatory cytokine to an intestinal permeation model using human intestinal cell cultivated strain Caco-2, to produce a state where intestinal barrier function can be collapsed in humans, and finding a substance capable of effectively ameliorating the state. As a result, a flavan-3-ol polymer having a weight-average molecular weight in a specific range was found to exhibit an excellent intestinal barrier function improvement action.

That is, the present invention relates to the following composition for improving intestinal barrier function.

(1) A composition for improving intestinal barrier function, containing a flavan-3-ol polymer having a weight-average molecular weight of 4500 to 50000 as an active ingredient.

(2) The composition for improving intestinal barrier function according to the above (1), wherein the flavan-3-ol polymer has a weight-average molecular weight of 4500 to 20000.

(3) The composition for improving intestinal barrier function according to the above (1) or (2), wherein the flavan-3-ol polymer has a weight-average molecular weight of 9000 to 16000.

(4) The composition for improving intestinal barrier function according to any one of the above (1) to (3), wherein the flavan-3-ol polymer is contained in at least one grape-derived raw material selected from the group consisting of grape pulp, grape seed coat, and grape seed.

(5) The composition for improving intestinal barrier function according to any one of the above (1) to (4), wherein the composition for improving intestinal barrier function is an oral composition.

(6) The composition for improving intestinal barrier function according to the above (5), wherein the oral composition is a food or beverage, a pharmaceutical product, or a quasi-pharmaceutical product.

(7) The composition for improving intestinal barrier function according to any one of the above (1) to (6), wherein the composition is used for intestinal regulation by improving the intestinal barrier function.

(8) The composition for improving intestinal barrier function according to any one of the above (1) to (7), wherein the composition is used in order to prevent or relieve abdominal discomfort by improving the intestinal barrier function.

(9) The composition for improving intestinal barrier function according to any one of the above (1) to (8), wherein the composition is labeled as having an intestinal regulation action.

(10) A method for improving intestinal barrier function, the method including: administering a flavan-3-ol polymer having a weight-average molecular weight of 4500 to 50000 to a subject.

(11) Use of a flavan-3-ol polymer having a weight-average molecular weight of 4500 to 50000 for improving intestinal barrier function.

### Advantageous Effects of Invention

The use of a composition for improving intestinal barrier function according to the present invention makes it possible to improve intestinal barrier function. The present invention improves the intestinal barrier function, which makes it possible to contribute also to prevention or amelioration of conditions or diseases related to intestinal barrier dysfunction.

### BRIEF DESCRIPTION OF DRAWINGS

FIG. 1 is a flow chart showing a procedure of purifying a flavan-3-ol polymer from a grape seed extract.

FIG. 2 is a flow chart showing a procedure of purifying a flavan-3-ol polymer from an Indian date extract.

FIG. 3 shows a calibration curve.

FIG. 4 is a graph showing the evaluation results of an intestinal barrier function improvement effect of a flavan-3-ol polymer using Caco-2 cells.

FIG. 5 shows a schedule for a test of Example 3.

FIG. 6 shows graphs of results of examining the effect of a flavan-3-ol polymer on the pain threshold of the large intestine, the intestinal permeability of the large intestine, and the expression of Claudin-2 ((a) pain threshold, (b) intestinal permeability of the large intestine, (c) Claudin-2 relative expression level).

### DESCRIPTION OF EMBODIMENTS

A composition for improving intestinal barrier function according to the present invention contains a flavan-3-ol polymer having a weight-average molecular weight (Mw) of 4500 to 50000 as an active ingredient.

The flavan-3-ol polymer in the present invention contains flavan-3-ol as a structural unit, and is a dimer or higher polymer with 4-6 or 4-8 interflavan bonds formed by condensation or polymerization. Examples of the flavan-3-ol include catechin and epicatechin. The flavan-3-ol polymer is one of polyphenols, and is a compound referred to as condensed tannin. The flavan-3-ol polymer used in the present invention may be a mixture of two or more polymers having different degrees of polymerization. In one aspect, the flavan-3-ol polymer may have a galloyl group.

The flavan-3-ol polymer having a weight-average molecular weight of 4500 to 50000 has an excellent intestinal barrier function improvement action.

In terms of the intestinal barrier function improvement effect, the weight-average molecular weight of the flavan-3-ol polymer is preferably 5000 or more, more preferably 9000 or more, still more preferably 9400 or more, particularly preferably 10000 or more. The weight-average molecular weight is preferably 20000 or less, more preferably 16000 or less, still more preferably 15000 or less. The range may be any combination of any upper limit and any lower limit. In one aspect, in terms of the intestinal barrier function improvement effect, the weight-average molecular weight of the flavan-3-ol polymer is preferably 4500 to 20000, more preferably 4500 to 16000, still more preferably 5000 to 16000, yet still more preferably 9000 to 16000, particularly preferably 9400 to 15000, most preferably 10000 to 15000. The flavan-3-ol polymer having a weight-average molecular weight of 10000 to 15000 has a particularly high intestinal barrier function improvement effect. The number-average molecular weight (Mn) of the flavan-3-ol polymer is preferably 1900 or more, more preferably 2400 or more, still more preferably 2900 or more, particularly preferably 3000 or more. The number-average molecular weight (Mn) is preferably 10000 or less, more preferably 8000 or less, still more preferably 6000 or less, particularly preferably 3200 or less, most preferably 3100 or less. In one aspect, the number-average molecular weight (Mn) of the flavan-3-ol polymer is preferably 1900 to 10000, more preferably 2400 to 10000, still more preferably 2400 to 8000, yet still more preferably 2900 to 6000, particularly preferably 3000 to 3200, most preferably 3000 to 3100. In one another aspect, the number-average molecular weight of the flavan-3-ol polymer is preferably 1900 to 3200, more preferably 1900 to 3100. In one aspect, for example, it is preferable that the flavan-3-ol polymer has a weight-average molecular weight (Mw) of 4500 to 50000 and a number-average molecular weight (Mn) of 1900 to 10000. It is preferable that the flavan-3-ol polymer has a Mw of 4500 to 50000 and a Mn of 2400 to 10000. It is more preferable that the flavan-3-ol polymer has a Mw of 4500 to 20000 and a Mn of 2400 to 8000. It is still more preferable that the flavan-3-ol polymer has a Mw of 9000 to 16000 and a Mn of 2900 to 6000. It is yet still more preferable that the flavan-3-ol polymer has a Mw of 9400 to 15000 and a Mn of 3000 to 3200. It is particularly preferable that the flavan-3-ol polymer has a Mw of 10000 to 15000 and a Mn of 3000 to 3200. In one another aspect, it is also preferable that the flavan-3-ol polymer has a Mw of 4500 to 16000 and a Mn of 1900 to 6000. It is more preferable that the flavan-3-ol polymer has a Mw of 5000 to 16000 and a Mn of 1900 to 6000. In terms of a particularly high intestinal barrier function improvement effect, it is most preferable that the flavan-3-ol polymer has a Mw of 10000 to 15000 and a Mn of 3000 to 3100.

In one aspect, the flavan-3-ol polymer has a molecular weight distribution (weight-average molecular weight/number-average molecular weight) of preferably 1.0 to 5.0, more preferably 1.5 to 4.8, still more preferably 1.8 to 4.8, particularly preferably 2.5 to 4.7. The molecular weight distribution is represented by the ratio of the weight-average molecular weight to the number-average molecular weight. In terms of the intestinal barrier function improvement effect, the flavan-3-ol polymer in the present invention preferably has a molecular weight distribution (weight-average molecular weight/number-average molecular weight) within the above range.

The flavan-3-ol polymer used in the present invention is not limited by the derivation or the producing method. For example, a plant-derived flavan-3-ol polymer extracted from a plant may be used, and a flavan-3-ol polymer obtained by a synthetic method may be used. For example, the flavan-3-ol polymer can be obtained from plants such as grape and Indian date (Tamarindus indica L). In one aspect of the present invention, the flavan-3-ol polymer is preferably one derived from grape. More preferably, the flavan-3-ol polymer is at least one selected from the group consisting of one contained in (derived from) grape pulp, one contained in (derived from) grape seed coat, and one contained in (derived from) grape seed. Still more preferably, the flavan-3-ol polymer is derived from grape seed (for example, derived from a grape seed extract).

The flavan-3-ol polymer used in the present invention is an ingredient contained in a plant such as grape, and has few side effects and high safety even if the compound is ingested for a long period of time. The present invention can provide the composition for improving intestinal barrier function, the composition containing a substance having few side effects and high safety as an active ingredient even if the composition is ingested for a long period of time.

For example, when the flavan-3-ol polymer is extracted and purified from the grape seed, the grape seed is extracted by hydrous alcohol, and the obtained extract is then filtered and the alcohol is removed, followed by performing column purification, whereby the grape seed extract containing the flavan-3-ol polymer can be obtained. A flavan-3-ol polymer having a weight-average molecular weight within the above range can be obtained by fractionating and purifying a plant extract such as a grape seed extract according to a method described in Examples, for example.

The weight-average molecular weight and number-average molecular weight of the flavan-3-ol polymer are values in terms of catechin and polystyrene measured by size exclusion chromatography. The weight-average molecular weight and number-average molecular weight of the flavan-3-ol polymer can be measured according to the method described in Examples to be described later. By dividing the obtained number-average molecular weight by the molecular weight (290) of the catechin, the degree of polymerization can be calculated.

In the present invention, the “intestinal barrier function” means the function of protecting the intrusion (permeation) of foreign materials (for example, toxins such as endotoxin, inflammatory substances, and undigested products) into the body from the outside of intestinal epithelial cells (the inside of the intestine). The large intestine and the small intestine are contained in the intestine. A state where the intrusion of the foreign materials into the body from the outside of the intestinal epithelial cells is promoted as compared with the normal state is referred to as a state where the permeability of the foreign materials in the intestinal epithelial cells increases (rises). The “intestinal barrier function improvement” means both suppression of the increase in the permeability of the foreign materials in the intestinal epithelial cells and decrease in the permeability of the foreign materials in the intestinal epithelial cells. In the present invention, the “intestinal barrier function improvement” is also used to mean to include suppression of the decrease in the intestinal barrier function and enhancement of the decreased intestinal barrier function. For example, the intestinal barrier function is improved by normalizing or strengthening a tight junction bonding the intestinal epithelial cells together. In one aspect, the composition for improving intestinal barrier function according to the present invention may be used in order to improve the intestinal barrier function by normalizing or strengthening the tight junction in the intestinal epithelial cells.

The intestinal barrier function improvement effect is indicated by, for example, increase in the electrical resistance value (transepithelial electric resistance: TEER) of the intestinal epithelial cells or suppression of the decrease in the TEER. A substance increasing the TEER or suppressing the decrease thereof has the action of normalizing or strengthening the tight junction in the intestinal epithelial cells. The intestinal barrier function improvement effect is also indicated by decrease in the amount of a substance permeating from the intestinal side of the intestinal epithelial cells to the inside of the body. The person skilled in the art can select a specific method of evaluating the intestinal barrier function improvement effect depending on the purpose. For example, as shown in Examples to be described later, a method for measuring TEER using an intestinal permeation model using human intestinal epithelial cells (Caco-2 cells) can be used. Specifically, inflammatory cytokine (TNFα, IL-1β, IFNγ and the like) is added into Caco-2 monolayer cultured cells to produce a state where the intestinal barrier function can be collapsed in human. If the addition of a test substance suppresses the decrease in the TEER as compared with the case where the substance is not added, the test substance can be evaluated to have the intestinal barrier function improvement effect. As shown in Examples, the flavan-3-ol polymer having a weight-average molecular weight of 4500 to 50000 has a high effect of suppressing the decrease in the TEER due to the addition of inflammatory cytokine in an intestinal permeation model using Caco-2, and has a high intestinal barrier function improvement effect. The flavan-3-ol polymer having a weight-average molecular weight of 4500 to 50000 can normalize or strengthen the tight junction in the intestinal epithelial cells to improve the intestinal barrier function. It is a surprising finding that the flavan-3-ol polymers having the above specific weight-average molecular weight exhibit an excellent intestinal barrier function improvement effect.

As shown in Examples, the flavan-3-ol polymer had the action of preventing or ameliorating a symptom of stress-induced large intestine hyperalgesia due to the intestinal barrier function improvement action. The prevention or amelioration effect of abdominal discomfort is expected by preventing or ameliorating the large intestine hyperalgesia. Therefore, by improving the intestinal barrier function, the flavan-3-ol polymer having a weight-average molecular weight of 4500 to 50000 is useful, for example, for preventing or relieving the abdominal discomfort.

The composition for improving intestinal barrier function according to the present invention contains the flavan-3-ol polymer having a weight-average molecular weight of 4500 to 50000 as an active ingredient, to have an excellent intestinal barrier function improvement action. For this reason, the composition for improving intestinal barrier function according to the present invention is useful for preventing or ameliorating conditions or diseases against which improvement of the intestinal barrier function is effective, for example, conditions or diseases related to the intestinal barrier dysfunction. The intestinal barrier dysfunction includes deterioration of the intestinal barrier function. Examples of the conditions or diseases related to the intestinal barrier dysfunction include conditions or diseases caused by the intestinal barrier dysfunction, or conditions or diseases involving the intestinal barrier dysfunction. Examples of the conditions or diseases related to the intestinal barrier dysfunction include inflammatory bowel diseases, irritable bowel syndromes, systemic autoimmune diseases (rheumatoid arthritis, erythematosus and the like), allergies (food allergy, pollinosis and the like), and metabolic syndromes (obesity, type I or type II diabetes mellitus, hypertension, hyperlipidemia, non-alcoholic fatty liver diseases (NAFLD), arteriosclerosis and the like) (for example, Camilleri et al., Am, J Physiol Gastrointest Liver Physiol 303: G775-G785, 2012; Mu et al., Front. Immunol., Vol. 8, Article 598, 2017; Bischoff et, al., BMC Gastroenterology 2014 14:189).

More specific examples of symptoms of the conditions or diseases related to the intestinal barrier dysfunction include symptoms such as diarrhea, constipation, and discomfort (bloating, borborygmi, abdominal pain and the like) of the gut (abdominal part). The composition for improving intestinal barrier function according to the present invention has the action of improving the intestinal condition by improving the intestinal barrier function. Therefore, the composition for improving intestinal barrier function according to the present invention can control the intestinal function by the intestinal barrier function improvement, and is useful for preventing or ameliorating the symptom of the intestine as described above. In one aspect, the composition for improving intestinal barrier function according to the present invention may be used for intestinal regulation (in order to prevent or ameliorate, for example, diarrhea, constipation, and abdominal discomfort) by improving the intestinal barrier function. Among these, the composition for improving intestinal barrier function is preferably used in order to prevent or relieve the abdominal discomfort, and is useful also for preventing or relieving stress-induced abdominal discomfort. The intestinal barrier dysfunction is also associated with metabolic syndromes and the like (for example, the Bischoff et al., BMC Gastroenterology 2014 14:189 as described above). It is effective also in prevention or amelioration of the metabolic syndromes to improve the intestinal barrier function. Examples of the symptoms of the metabolic syndromes include abnormal glucose metabolism, abnormal lipid metabolism, increase in body fat, increase in visceral fat, increase in abdominal circumference fat, and higher blood pressure. Therefore, the composition for improving intestinal barrier function according to the present invention can improve the intestinal barrier function to contribute to improvement of glucose metabolism, improvement of lipid metabolism, decrease or suppression of increase in fat such as body fat, visceral fat, or abdominal circumference fat, amelioration of higher blood pressure and the like.

Herein, the “prevention of conditions or diseases” refers to enhancement of the resistance of a subject to the conditions or diseases, or delay or prevention of the onset of the conditions or diseases. Herein, the “amelioration of conditions or diseases” refers to recovery of a subject from the conditions or diseases, alleviation of the symptoms of the conditions or diseases, or delay or prevention of the progress of the conditions or diseases.

The composition of the present invention is applicable for both therapeutic use (medical use) and non-therapeutic use (non-medical use).

The composition for improving intestinal barrier function according to the present invention can be provided, for example, as a food or beverage, a pharmaceutical product, a quasi-pharmaceutical product, feed or the like, but it is not limited thereto. The composition for improving intestinal barrier function according to the present invention may be a food or beverage, a pharmaceutical product, a quasi-pharmaceutical product, or feed by itself, and may be a formulation or material such as an additive used therefor. The composition for improving intestinal barrier function according to the present invention may be provided as an agent as one example, but it is not limited thereto. The agent may be provided as a composition by itself or as a composition containing the agent.

In one aspect, the composition for improving intestinal barrier function according to the present invention is preferably an oral composition. The present invention can provide an oral composition having an excellent intestinal barrier function improvement action. Examples of the oral composition include a food or beverage, a pharmaceutical product, and a quasi-pharmaceutical product, and the oral composition is preferably a food or beverage.

The composition for improving intestinal barrier function according to the present invention may contain one or two or more ingredients (other ingredients) other than the above-described flavan-3-ol polymer as long as the effect of the present invention is not impaired. In one aspect, for example, ingredients such as lactic acid bacteria, bifidobacteria, dietary fibers, and polysaccharides may be contained as the other ingredients.

It is preferable that the lactic acid bacteria and the bifidobacteria can be orally ingested.

The dietary fiber may be any of a water-insoluble dietary fiber and a water-soluble dietary fiber. Examples of the water-insoluble dietary fiber include cellulose, lignin, hemicellulose, wheat bran, an apple fiber, a sweet potato fiber, and chitin. The water-soluble dietary fiber is roughly divided into a high viscous dietary fiber and a low viscous dietary fiber, and examples of the high viscous dietary fiber include pectin, konjac mannan, alginic acid, sodium alginate, guar gum, and agar. Among the dietary fibers generally known in Japan, the low viscous water-soluble dietary fiber is referred to as a dietary fiber material containing 50% by weight or more of a dietary fiber and dissolved in ordinary temperature water to provide a low viscous solution, i.e., an about 5% by weight aqueous solution having a viscosity of 20 mPa·s or less. Examples of the low viscous water-soluble dietary fiber include hardly digestible dextrin, polydextrose, partially hydrolyzed guar gum, and Litesse (polydextrose). Another examples of the low viscous water-soluble dietary fiber include dietary fiber materials satisfying low viscous and water-soluble properties. One of the dietary fibers may be used, and two or more thereof may be used.

Examples of the polysaccharides include oligosaccharides such as galactooligosaccharides, xylooligosaccharides, mannooligosaccharides, agarooligosaccharides, fructooligosaccharides, isomaltooligosaccharides, and raffinose. One of the oligosaccharides may be used, and two or more thereof may be used.

The composition for improving intestinal barrier function according to the present invention may contain optional additives and optional ingredients other than the above. The additives and the ingredients may be selected depending on the form of the composition for improving intestinal barrier function, and the like. The additives and the ingredients generally usable for a food or beverage, a pharmaceutical product, a quasi-pharmaceutical product, feed or the like can be used. Examples thereof include various additives which are acceptable in a food or beverage or pharmaceutically acceptable as an oral administration agent, such as an excipient, a lubricant, a stabilizer, a dispersant, a binder, a diluent, a flavour, a sweetener, a flavoring agent, and a colorant. For example, when the composition for improving intestinal barrier function according to the present invention is used as the oral composition, the oral composition can appropriately contain ingredients capable of being orally ingested other than the above as long as the effect of the present invention is not impaired. Such ingredients include vitamin, a vitamin-like substance, protein, amino acid, fat and oil, organic acid, a carbohydrate, a plant-derived raw material, an animal-derived raw material, a microorganism, an additive for food or beverage, and an additive for pharmaceutical product.

In addition to the above, ingredients such as materials used for a food or beverage, a pharmaceutical product, a quasi-pharmaceutical product, feed or the like can be appropriately blended depending on the use.

The form of the composition for improving intestinal barrier function according to the present invention is not limited as long as the effect of the present invention is obtained. Examples of the form include tablets, pills, granules, fine granules, chews, capsules (including soft and hard capsules), liquids, chewable tablets, and beverages. The form may be other food form. These dosage forms may be prepared by using conventional methods commonly known in the art.

In one aspect, when the composition for improving intestinal barrier function according to the present invention is used as a food or beverage, ingredients (for example, a material for food or beverage, and an additive used as necessary) usable for the food or beverage can be blended with the flavan-3-ol polymer to provide various foods or beverages (compositions for food or beverage). The food or beverage is not limited. Examples thereof include general foods and beverages, health foods, foods with function claims, foods for specified health uses, foods for the sick, food additives, and raw materials thereof. The form of the food or beverage is not also limited, and examples thereof include various formulation forms such as solid oral formulations (such as tablets, coating tablets, fine granules, granules, powders, pills, capsules (including soft and hard capsules), dry syrup agents, and chewable tablets); and liquid oral formulations (such as internal liquid formulations and syrups). In one aspect of the present invention, the food or beverage may contain one or two or more of the lactic acid bacteria, the bifidobacteria, the dietary fibers, and the polysaccharides.

When the composition for improving intestinal barrier function according to the present invention is used as the pharmaceutical product or the quasi-pharmaceutical product, an additive such as a pharmaceutically acceptable excipient can be blended with the flavan-3-ol polymer, to provide the pharmaceutical products (pharmaceutical compositions) or quasi-pharmaceutical products (quasi-pharmaceutical product compositions) of various dosage forms. The form of administration of the pharmaceutical product or quasi-pharmaceutical product is preferably oral administration. The dosage form of the pharmaceutical product or quasi-pharmaceutical product may be a dosage form suitable to the form of administration. Examples of the dosage form of the oral pharmaceutical product or quasi-pharmaceutical product include solid oral formulations such as tablets, coating tablets, fine granules, granules, powders, pills, capsules (including soft and hard capsules), dry syrup agents, and chewable tablets; and liquid oral formulations such as internal liquid formulations and syrups.

The tablets, the pills, and the granules may be in dosage forms conventionally coated as necessary such as sugar-coated tablets, gelatin-coated preparations, enteric-coated preparations, and film-coated agents. The tablets may be in the form of double or multiple layer tablets.

When the composition for improving intestinal barrier function according to the present invention is used as a food or beverage, a pharmaceutical product, a quasi-pharmaceutical product, feed or the like, the producing method is not limited, and the composition for improving intestinal barrier function can be produced by a general method using the flavan-3-ol polymer. The present invention also includes use of the flavan-3-ol polymer having a weight-average molecular weight of 4500 to 50000 for the manufacture of the composition for improving intestinal barrier function.

The composition for improving intestinal barrier function according to the present invention may be labeled with one or two or more of the following information on a package, container, or package insert: usage, types of active ingredients, the above-described effects, and instructions for use (e.g., ingestion method or administration method). The composition for improving intestinal barrier function according to the present invention may be labeled as having an intestinal barrier function improvement action or an action based on the intestinal barrier function improvement action. The composition for improving intestinal barrier function may be labeled as having an intestinal regulation action, for example.

The intestinal regulation action may be an intestinal regulation action based on improvement of intestinal barrier function, and is not limited. Examples of the labeling as having an intestinal regulation action include “for one tending to be constipated or have diarrhea”, “for one worrying about gut function”, “for one readily feeling discomfort of gut”, “improvement of bowel movement”, “improvement of stool state”, “amelioration of defecation frequency”, “amelioration of defecation output”, “gut feels better”, “controlling gut function”, “controlling intestinal function”, “amelioration of discomfort in gut”, “reduction of generation of gas”, “reduction of abdominal bloatedness”, and “amerilation of borborygmi”. The composition for improving intestinal barrier function according to the present invention may include one or two or more of such labelings.

The content of the flavan-3-ol polymer having a weight-average molecular weight of 4500 to 50000 in the composition for improving intestinal barrier function according to the present invention can be appropriately set depending on the form of the composition and the like. In one aspect, when the composition for improving intestinal barrier function is used as the oral compositions such as a food or beverage, a pharmaceutical product, and a quasi-pharmaceutical product, the content of the flavan-3-ol polymer is preferably 0.0001% by weight or more, more preferably 0.01% by weight or more in the composition. The content of the flavan-3-ol polymer is preferably 80.0% by weight or less, more preferably 20.0% by weight or less in the composition. In one aspect, the content of the flavan-3-ol polymer is preferably 0.0001 to 80.0% by weight, more preferably 0.01 to 20.0% by weight in the composition for improving intestinal barrier function.

The content of the flavan-3-ol polymer can be measured according to known methods, and for example, an HPLC method can be used.

The composition for improving intestinal barrier function according to the present invention can be ingested or administered by a suitable method depending on the form. It is preferable that the composition for improving intestinal barrier function according to the present invention is orally administered or orally ingested.

The amount of the composition for improving intestinal barrier function according to the present invention ingested (which may also be referred to as the amount administered) is not limited, and may be an amount such that the intestinal barrier function improvement effect is obtained. The amount ingested may be appropriately set according to the form of administration or administration method, for example. As one aspect, for the amount of the composition for improving intestinal barrier function ingested when the composition is orally administered to or allowed to be ingested by a human (adult) subject (for example, weight: 60 kg), the amount of the flavan-3-ol polymer having a weight-average molecular weight of 4500 to 50000 ingested is preferably 1 to 2000 mg, more preferably 10 to 1500 mg, still more preferably 30 to 1000 mg, particularly preferably 100 to 1000 mg per day. It is preferable that the amount is orally administered or allowed to be ingested, for example, once daily or in a divided manner two to three times daily. When the composition for improving intestinal barrier function is allowed to be ingested by the human (adult) subject for the purpose of obtaining the intestinal barrier function improvement effect, the composition for improving intestinal barrier function is preferably allowed to be ingested by or administered to the subject so that the amount of the flavan-3-ol polymer ingested is within the above range. In one aspect, the composition for improving intestinal barrier function according to the present invention may be an oral composition for allowing the adult to ingest or administering the above amount of the flavan-3-ol polymer per day per weight of 60 kg to the adult.

In one aspect, the composition for improving intestinal barrier function according to the present invention preferably contains the flavan-3-ol polymer in an amount such that the desired effect of the present invention is obtained, that is, an effective amount in consideration of the form of administration, administration method and the like thereof. As one aspect, for example, when the composition for improving intestinal barrier function is an oral composition such as a food or beverage or an oral pharmaceutical product, the content of the flavan-3-ol polymer in the amount of the composition ingested per adult (for example, weight: 60 kg) per day is preferably 1 to 2000 mg, more preferably 10 to 1500 mg, still more preferably 30 to 1000 mg, particularly preferably 100 to 1000 mg.

A greater intestinal barrier function improvement effect can be expected by continuous ingestion (administration) of the flavan-3-ol polymer which is an active ingredient of the composition for improving intestinal barrier function according to the present invention. In a preferable aspect, the composition for improving intestinal barrier function according to the present invention is continuously ingested. In one embodiment of the present invention, it is preferable that the composition for improving intestinal barrier function is continuously ingested for a week or more.

A subject (hereinafter, merely referred to as an “administration subject”) to whom the composition for improving intestinal barrier function according to the present invention is administered or by whom the composition is allowed to be ingested is preferably human or non-human animal, more preferably mammal (human or non-human mammal), still more preferably human. The administration subject in the present invention is preferably a subject requiring or desiring the intestinal barrier function improvement. Suitable examples of the subject include a subject having a reduced intestinal barrier function and a subject desiring the prevention or amelioration of conditions or diseases related to intestinal barrier dysfunction.

The present invention also includes the following method for improving intestinal barrier function and the like.

A method for improving intestinal barrier function, the method including: administering the flavan-3-ol polymer having a weight-average molecular weight of 4500 to 50000 to a subject:

Use of the flavan-3-ol polymer having a weight-average molecular weight of 4500 to 50000 for improving intestinal barrier function.

The method and the use may be therapeutic or non-therapeutic. The “non-therapeutic” is a concept which does not include medical activities, i.e., a concept which does not include surgery, therapy or diagnosis.

The amount of the flavan-3-ol polymer administered may be an amount such that the intestinal barrier function improvement effect is obtained, i.e., an effective amount, and is not limited. For example, the above-described amount is preferably administered. An administration route is preferably oral administration. The flavan-3-ol polymer may be administered as it is, or a composition containing the above-described flavan-3-ol polymer may be administered. For example, the above-described composition for improving intestinal barrier function according to the present invention can be administered. The flavan-3-ol polymer, the subject (administration subject), the administration method, the amount administered, preferable aspects thereof and the like are the same as those in the above-described composition for improving intestinal barrier function. In one aspect, when the flavan-3-ol polymer is orally administered to, for example, a human (adult) subject, the daily amount of the flavan-3-ol polymer administered is preferably 1 to 2000 mg, more preferably 10 to 1500 mg, still more preferably 30 to 1000 mg, particularly preferably 100 to 1000 mg. In the above method and use, it is preferable that the flavan-3-ol polymer is continuously administered for a week or more.

### EXAMPLES

The following provides Examples which more specifically describe the present invention. The present invention is not limited to these Examples.

### Example 1

### Purification of Flavan-3-Ol Polymer

A flavan-3-ol polymer (hereinafter, the flavan-3-ol polymer is also referred to as OPC) was purified from a grape seed extract or an Indian date (Tamarindus indica L) extract.

The OPC was fractionated and purified by a method described in Biosci. Biotechnol. Biochem., 73(6), 1274-1279 (2009). A commercially available grape seed extract having an amount of OPC of 81% or more, and an Indian date extract having an amount of OPC of 35% or more as a standard were used as starting materials.

A specific procedure of purifying the OPC from the grape seed extract will be described later. A transition rate (%) means “100×yield (g)/starting material (g).”

A grape seed extract (10.2 g) was dissolved in water (100 mL), and then subjected to liquid-liquid separation three times with ethyl acetate (100 mL). An ethyl-acetate layer was concentrated to dryness to obtain a fraction 1 (Fr.1) (transition rate: 26%). The water layer was concentrated to dryness to obtain a fraction 2 (Fr.2) (transition rate: 72%). The fraction 2 was dissolved in methanol (120 mL), and chloroform (80 mL) was added thereto. The obtained solution was centrifuged (3000 rpm, 5 minutes) to be separated into a precipitate (P1) and a supernatant (S1). The precipitate (P1) was concentrated to dryness to obtain a fraction 3 (Fr.3) (transition rate: 17%).

Chloroform (40 mL) was added to the supernatant (S1), followed by centrifugal separation (3000 rpm, 5 minutes), to be separated into a precipitate (P2) and a supernatant (S2). The precipitate (P2) was concentrated to dryness to obtain a fraction 4 (Fr.4) (transition rate: 10%).

Chloroform (60 mL) was added to the supernatant (S2), followed by centrifugal separation (3000 rpm, 5 minutes), to be separated into a precipitate (P3) and a supernatant (S3). The precipitate (P3) was concentrated to dryness to obtain a fraction 5 (Fr.5) (transition rate: 13%).

Chloroform (100 mL) was added to the supernatant (S3), followed by centrifugal separation (4000 rpm, 5 minutes), to be separated into a precipitate (P4) and a supernatant (S4).

The precipitate (P4) was concentrated to dryness to obtain a fraction 6 (Fr.6) (transition rate: 12%). The supernatant (S4) was concentrated to dryness, to obtain a fraction 7 (Fr.7) (transition rate: 18%).

A specific procedure of purifying an OPC from the Indian date extract will be described later.

The Indian date extract (10.2 g) was dissolved in water (100 mL), and then subjected to liquid-liquid separation three times with ethyl acetate (100 mL). An ethyl-acetate layer was concentrated to dryness to obtain a fraction 8 (Fr.8) (transition rate: 2%). A water layer was concentrated to dryness to obtain a fraction 9 (Fr.9) (transition rate: 91%). The fraction 9 was dissolved in methanol (160 mL), and chloroform (40 mL) was added thereto. The obtained solution was centrifuged (3000 rpm, 5 minutes) to be separated into a precipitate (ppt) and a supernatant. The precipitate was concentrated to dryness to obtain a fraction 10 (Fr.10) (transition rate: 59%), and the supernatant was concentrated to dryness to obtain a fraction 11 (Fr.11) (transition rate: 28%).

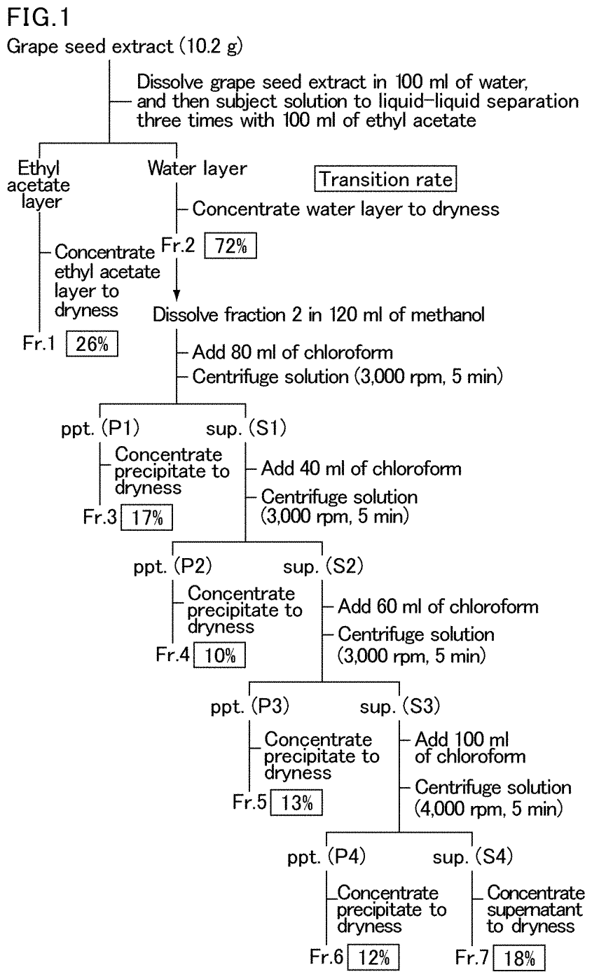

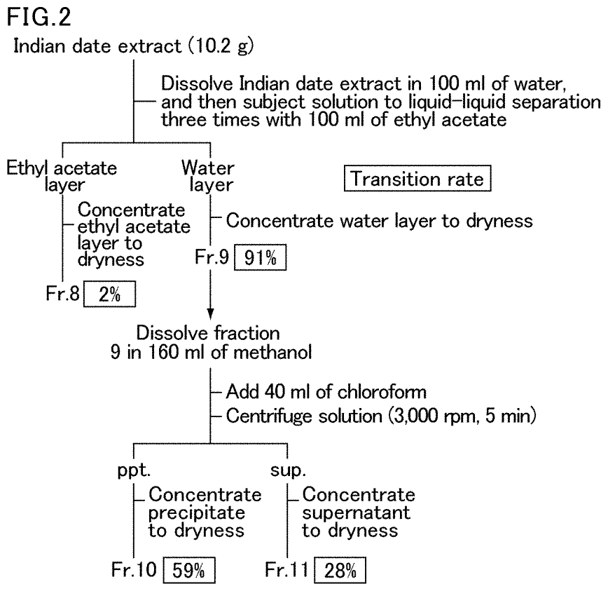

FIG. 1 is a flow chart showing a procedure of purifying each fraction from the grape seed extract. FIG. 2 is a flow chart showing a procedure of purifying each fraction from the Indian date extract. In FIG. 1 and FIG. 2, ppt. and sup. mean the precipitate and the supernatant, respectively.

### (Measurement of Weight-Average Molecular Weight and Number-Average Molecular Weight of Flavan-3-Ol Polymer)

The weight-average molecular weight and number-average molecular weight of the OPC of each fraction (OPC fraction) prepared above were measured using high-performance liquid chromatography (HPLC) under analysis conditions to be described below. Analysis soft LC Solution GPC manufactured by Shimadzu Corporation was used for calculating the weight-average molecular weight and the number-average molecular weight. As a calibration curve, a cubic curve was drawn using standard polystyrene samples SL-105 (Lot. 00301 manufactured by Shodex) having molecular weights of 780, 2340, 6180, and 20000, and (+)-catechin hydrate (C1251 manufactured by Sigma-Aldrich Co. LLC.).

### (HPLC Analysis Conditions)

### Detector: UV (261 nm)

### Column: Shodex OHpak SB-806MHQ (ϕ7.6×25 mm)

### Shodex OHpak SB-802.5HQ (ϕ7.6×250 mm)

(Guard column: Shodex OHpak SB-G)

### Solvent: 20 mM LiBr/dimethylformamide (DMF)

Column temperature: 40° C.
Flow rate: 0.6 mL/min

An evaluation sample was dissolved in 20 mM LiBr/DMF so that the concentration was set to 50 mg/mL, and 10 μL of the solution was then injected.

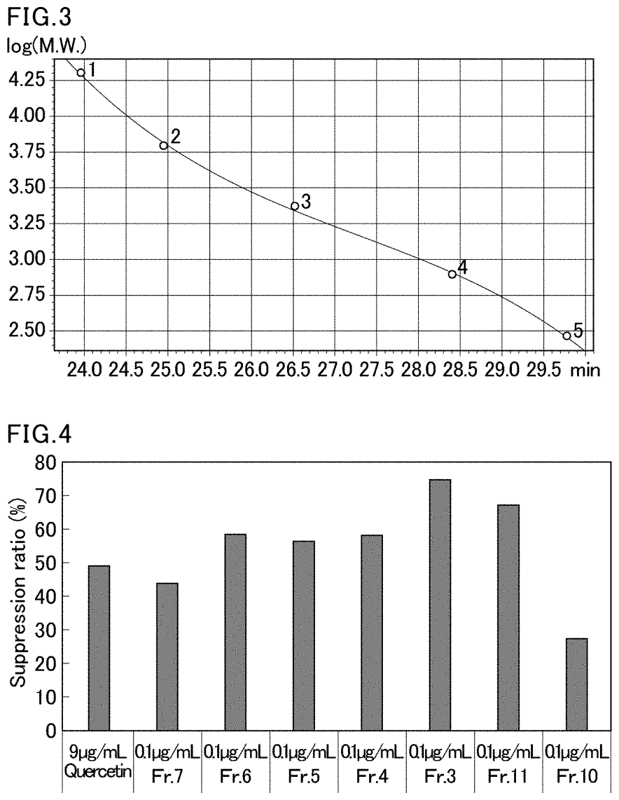

FIG. 3 shows the prepared calibration curve. In FIG. 3, numeral number 1 designates molecular weight 20000 (23.967 minutes); numeral number 2 designates molecular weight 6180 (24.955 minutes); numeral number 3 designates molecular weight 2340 (26.527 minutes); numeral number 4 designates molecular weight 780 (28.409 minutes); and numeral number 5 designates molecular weight 290 (29.781 minutes). A third-order formula (regression formula) determined from the calibration curve shown in FIG. 3 is shown below.

Y=aX³+bX²+cX+d

a=−0.01074954, b=0.8799255, c=−24.22844, d=227.5189

The grape seed extract and Indian date extract used for the raw materials, and the fractions 1 to 11 (Fr.1 to 11) obtained by purification were subjected to the present analysis to calculate the purity, weight-average molecular weight, and number-average molecular weight of the OPC. These are shown in Table 1.

TABLE 1

| | Weight-average | | Number-average | | |
|---|---|---|---|---|---|
| | molecular weight of | Average degree of | molecular weight of | Average degree of | Peak purity of OPC |
| | OPC | polymerization 1 | OPC | polymerization 2 | (%) |
| Grape seed extract | 5633 | 20 | 2319 | 8 | 89.7 |
| Fr. 1 | 2510 | 9 | 1264 | 4 | 65.5 |
| Fr. 2 | 6682 | 24 | 2793 | 10 | 97.9 |
| Fr. 3 | 14107 | 50 | 3029 | 11 | 100.0 |
| Fr. 4 | 9422 | 33 | 3169 | 11 | 100.0 |
| Fr. 5 | 7000 | 25 | 2883 | 10 | 100.0 |
| Fr. 6 | 4611 | 16 | 2466 | 9 | 100.0 |
| Fr. 7 | 2778 | 10 | 1603 | 6 | 95.5 |
| Indian date extract | 28973 | 102 | 7463 | 26 | 99.7 |
| Fr. 8 | 2294 | 8 | 1316 | 5 | 86.9 |
| Fr. 9 | 24380 | 86 | 7126 | 25 | 100.0 |
| Fr. 10 | 52211 | 184 | 12468 | 44 | 100.0 |
| Fr. 11 | 15798 | 56 | 5796 | 20 | 99.8 |

In Table 1, an average degree of polymerization 1 is determined by dividing the weight-average molecular weight of the OPC by the molecular weight 290 of catechin. An average degree of polymerization 2 is determined by dividing the number-average molecular weight of the OPC by the molecular weight 290 of catechin.

The peak purity of the OPC is expressed in area percentage of the peak area of the OPC, taking the total area of peaks detected in a HPLC (UV 261 nm) chromatogram as 100%.

As for the molecular weight distribution (weight-average molecular weight/number-average molecular weight) of the OPC of each fraction, Fr.1 was 2.0; Fr.2 was 2.4; Fr.3 was 4.7; Fr.4 was 3.0; Fr.5 was 2.4; Fr.6 was 1.9; Fr.7 was 1.7; Fr.8 was 1.7; Fr.9 was 3.4; Fr.10 was 4.2; and Fr.11 was 2.7.

### Example 2

### Evaluation of Ingredient Improving Intestinal Barrier Function Using Caco-2 Cells

Caco-2 cells were cultured for three weeks at 37° C. in Transwell (manufactured by Millicell Corporation) using DMEM (Dulbecco's modified Eagle's medium). A medium was removed from a plate of cultured Caco-2 cells, and the well was washed 3 times with serum-free DMEM. The well was filled with the medium. Then, the transepithelial electrical resistance (TEER) of Caco-2 monolayer cells was measured by Millicell-ERS (manufactured by Millipore Corporation). Cells (TEER 1000 Ω·cm²) determined that a sufficient tight junction was formed were selected, and used for the next screening. Then, to test solutions (media) on both apical and basolateral membrane sides, a test sample (hereinafter, merely referred to as a sample), TNFα (40 ng/mL), IL-1β (20 ng/mL), and IFNγ (10 ng/mL) were added, followed by cultivating for 48 hours. The sample was dissolved in dimethyl sulfoxide (DMSO), and the solution was then added to the test solutions. In this case, a well to which inflammatory cytokine (TNFα, IL-1β, and IFNγ) and the sample were not added was provided as normal. A well to which inflammatory cytokine was added and the sample was not added was provided as control. After the cultivation, TEER was measured again, to evaluate whether the sample suppressed the reduction (decrease) of TEER due to the inflammatory cytokine.

The TEER reduction suppression ratio (%) due to the sample was determined according to the following formula from the TEER values of the well to which the sample was added, normal, and control.

(calculating formula of TEER reduction suppression ratio)

TEER reduction suppression ratio (%)=100×((TEER of well to which sample is added)−(TEER of control))/((TEER of normal)−(TEER of control))

In this evaluation system, as the TEER reduction suppression ratio (%) is higher, an intestinal barrier function improvement effect is higher.

Each of fractions of Fr.3 to 7, 10, and 11 prepared in Example 1 was used for the sample. The sample was added into the test solution so that the concentration of the OPC in the test solution was set to 0.1 μg/mL. With respect to the amount of the sample used, the concentration was corrected by the peak area of the OPC determined in the measurement of the degree of polymerization. The results are shown in FIG. 4.

For comparison, quercetin for which a tight junction barrier function improvement effect has been reported (Non-Patent Literature 1) was subjected to the above evaluation. In the above test, 9 μg/mL of quercetin (Funakoshi Co., Ltd.) as the sample was added into the test solution, to evaluate the TEER reduction suppression ratio.

FIG. 4 is a graph showing the evaluation results of an intestinal barrier function improvement effect of a flavan-3-ol polymer using Caco-2 cells. The “suppression ratio (%)” in FIG. 4 means the TEER reduction suppression ratio (%).

The OPC of each of Fr.3 to 6 and 11 is a flavan-3-ol polymer having a weight-average molecular weight of 4500 to 50000 and used in the present invention. The OPC of each of Fr.7 and 10 is outside the scope of the present invention without having a weight-average molecular weight within the above range.

Fr.3 to 6 and 11 had a superior effect of suppressing TEER reduction to that of Fr.7 and 10, to have an excellent intestinal barrier function improvement action. Fr.3 to 6 and 11 exhibited a superior intestinal barrier function improvement action at a lower concentration to that of quercetin.

### Example 3

### Effect of Flavan-3-Ol Polymer (OPC) on Intestinal Permeability and Stress-Related Large Intestine Hyperalgesia

A series of animal experiments was performed based on a plan approved by the relevant chief through evaluation of the in-house animal experiment committee, in compliance with the animal welfare management laws and other related laws and regulations.

### (Method)

A commercially available grape seed extract containing 83% of OPC was used for administering a flavan-3-ol polymer (OPC). The weight-average molecular weight and number-average molecular weight of the flavan-3-ol polymer which was contained in the grape seed extract were analyzed by the method described in Example 1. The number-average molecular weight, weight-average molecular weight, and molecular weight distribution (weight-average molecular weight/number-average molecular weight) of the OPC were 1971, 5139, and 2.6, respectively.

Male Sprague-Dawley rats weighing about 300 g were divided into three groups.

Group 1: no stress+vehicle (N=7), Group 2: stress+vehicle (N=6), Group 3: stress+grape seed extract (N=6)

The grape seed extract was administered to the rats of group 3, and vehicle (distilled water) was administered to the rats of groups 1 and 2.

The vehicle or the grape seed extract was orally administered to the rats for one week (once daily) before the rats were subjected to stress. A solution of 10 mg/mL in which the grape seed extract was dissolved in distilled water was administered. The amount of the grape seed extract administered was set to 83 mg/kg (10 mL/kg) per day per weight in terms of the OPC. 10 mL/kg of distilled water was administered per weight to the rats of groups 1 and 2. During the test period, the rats were fed with free access to feed (CRF-1, Oriental Yeast Co., ltd.) and water.

The vehicle or the grape seed extract was administered to the rats for one week as described above, and the rats of groups 2 and 3 were then subjected to a water avoidance stress (WAS) load.

The rats were placed on a platform located at the center of a water tank for 1 hour for a stress treatment for three days (once daily) to induce large intestine hyperalgesia. During the stress treatment period (three days), the vehicle or the grape seed extract was administered 1.5 hours before the rats were subjected to stress.

The large intestine hyperalgesia was evaluated by the following method. Under anesthesia, a balloon catheter (silicone catheter, 2.0 mm, Terumo Corporation) was transanally inserted 2 cm into the rat, and an electrode (Teflon (registered trademark)-coated stainless steel, 0.05 mm, MT Research Institute, Inc.) was inserted 2 mm into left side exterior oblique muscles. Then, the rat was awakened in a state where it was placed in a ballman cage. To induce large intestine pain, the balloon catheter was gradually enlarged by pouring water after 30 minutes of treatment. Abdominal muscle contraction induced by large intestine pain was observed by using an electromyogram, and a pain threshold was measured. The pain threshold was measured twice before and after stress sessions. The pre-stress measurement was performed immediately before the stress treatment (before stress) on the first day. In the measurement after stress, a balloon catheter and an electrode were inserted into the rats as described above 24 hours after the last stress treatment (the third day of the stress treatment), and a pain threshold was measured after 30 minutes. The vehicle or the grape seed extract was orally administered 1.5 hours before the pain threshold was measured after stress.

In the evaluation results of the pain threshold, the pain threshold before stress was taken as 100%, and change in the pain threshold after stress (100×(pain threshold after stress)/(pain threshold before stress)) (%) was calculated.

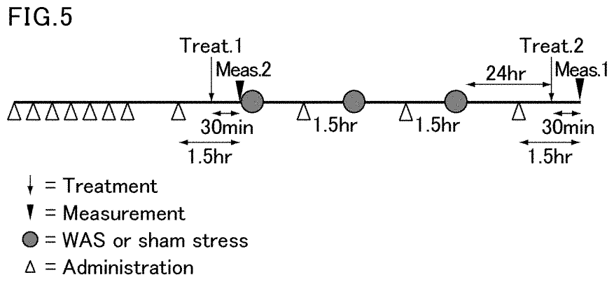

A test schedule of Example 3 is shown in FIG. 5. In FIG. 5, an arrow (1) represents surgery in which a balloon catheter is inserted into a rat; an inverse triangle (▾) represents measurement of a pain threshold; a circle (•) represents WAS (water avoidance stress) or no stress (sham stress); and a triangle (Δ) represents administration of the grape seed extract or vehicle. Treat.1 and Meas.1 represent the insertion surgery of the balloon catheter and the measurement of the pain threshold before stress, respectively. Treat.2 and Meas.2 represent the insertion surgery of the balloon catheter and the measurement of the pain threshold after stress, respectively.

After the evaluation of the large intestine hyperalgesia, the intestinal permeability of the large intestine and the expression level of a tight junction protein were measured by the following method.

The large intestine hyperalgesia after stress was evaluated, and the contents of the large intestine were then washed under anesthesia. The upper part of the large intestine was ligated at two points, to produce a 4 cm loop. 1 mL of a 1.5% Evans blue solution was injected thereto, and allowed to stand for 15 minutes. The ligation site was taken out, and washed with PBS and N-acetyl-cysteine. Evans blue permeating was extracted with 2 mL of N,N-dimethylformamide. Then, the absorbance was measured to determine the amount of Evans blue permeating. The intestinal permeability (mg/g tissue) of the large intestine was calculated by correcting the amount (mg) of Evans blue permeating by the weight (g) of the ligation site of the large intestine.

The expression level of tight junction protein Claudin-2 was analyzed by the Wes system of ProteinSimple. A 1-cm tissue was extracted from under the ligation site of the large intestine, and a sample was prepared using a tissue dissolving solution (1% SDS, 1% Triton, 1% sodium deoxycholate in PBS).

### (Results)

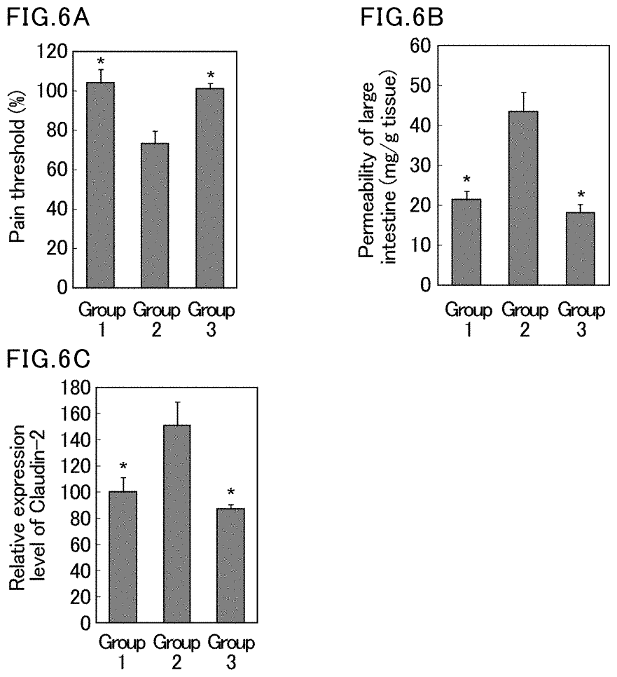

FIGS. 6(a) to 6(c) show the results of examining the effect of the flavan-3-ol polymer on the pain threshold of the large intestine, the intestinal permeability of large intestine, and the expression of Claudin-2, respectively. FIG. 6(a) shows the evaluation results of the pain threshold; FIG. 6(b) shows the evaluation results of the intestinal permeability of the large intestine (permeability of the large intestine); and FIG. 6(c) shows the relative expression level of Claudin-2. The relative expression level of Claudin-2 is a relative amount when the expression level in group 1 is taken as 100. Each graph shows mean standard error. To analyze a statistically significant difference between the groups, Dunnett test was performed against Group 2 (stress+vehicle group) (*: p<0.05).

The pain threshold was decreased by the stress treatment. The pain threshold was increased by administering the OPC. The intestinal permeability was increased by the stress treatment, and the OPC suppressed or ameliorated the increase in the intestinal permeability. The expression level of the tight junction protein Claudin-2 was increased by the stress treatment, and the OPC suppressed or ameliorated the increase in the expression level. The increase in the expression level of Claudin-2 has been known to cause an increase in intestinal permeability. The increase in intestinal permeability, and the increase in the expression level of Claudin-2 suggested the decrease in the tight junction function of the intestinal epithelial cells due to stress. The OPC had the action of improving the intestinal barrier function.

### INDUSTRIAL APPLICABILITY

A composition for improving intestinal barrier function according to the present invention is useful in the food or beverage field, the medicine field and the like.

1. A composition for improving intestinal barrier function, comprising a flavan-3-ol polymer having a weight-average molecular weight of 4500 to 50000 as an active ingredient.

2. The composition for improving intestinal barrier function according to claim 1,
wherein the flavan-3-ol polymer has a weight-average molecular weight of 4500 to 20000.

3. The composition for improving intestinal barrier function according to claim 1,
wherein the flavan-3-ol polymer has a weight-average molecular weight of 9000 to 16000.

4. The composition for improving intestinal barrier function according to claim 1,
wherein the flavan-3-ol polymer is contained in at least one grape-derived raw material selected from the group consisting of grape pulp, grape seed coat, and grape seed.

5. The composition for improving intestinal barrier function according to claim 1,
wherein the composition for improving intestinal barrier function is an oral composition.

6. The composition for improving intestinal barrier function according to claim 5,
wherein the oral composition is a food or beverage, a pharmaceutical product, or a quasi-pharmaceutical product.

7. The composition for improving intestinal barrier function according to claim 1,
wherein the composition is used for intestinal regulation by improving the intestinal barrier function.

8. The composition for improving intestinal barrier function according to claim 1,
wherein the composition is used in order to prevent or relieve abdominal discomfort by improving the intestinal barrier function.

9. The composition for improving intestinal barrier function according to claim 1,
wherein the composition is labeled as having an intestinal regulation action.

10. A method for improving intestinal barrier function, the method comprising:
administering a flavan-3-ol polymer having a weight-average molecular weight of 4500 to 50000 to a subject.

11. Use of a flavan-3-ol polymer having a weight-average molecular weight of 4500 to 50000 for improving intestinal barrier function.
